(19) United States
(12) Patent Application Publication
(10) Pub. No.: US 2015/0010641 A1
(43) Pub. Date: Jan. 8, 2015

(54) MICRO-, SUBMICRO- AND NANO-STRUCTURES CONTAINING AMARANTH PROTEIN

(71) Applicants: CONSEJO SUPERIOR DE INVESTIGACIONES CIENTIFICAS (CSIC), Madrid (ES); UNIVERSIDAD AUTONOMA DE QUERETARO, Queretaro (MX)

(72) Inventors: Amparo Lopez Rubio, Paterna (ES); Jose Maria Lagaron Cabello, Paterna (ES); Marysol Aceituno Medina, Paterna (ES); Sandra Mendoza Diaz, Queretaro (MX)

(73) Assignees: CONSEJO SUPERIOR DE INVESTIGACIONES CIENTIFICAS (CSIC), Madrid (ES); UNIVERSIDAD AUTONOMA DE QUERETARO, Queretaro (MX)

(21) Appl. No.: 14/353,636

(22) PCT Filed: Oct. 17, 2012
(86) PCT No.: PCT/ES2012/070719

§ 371 Date: Apr. 23, 2014

(30) Foreign Application Priority Data
Oct. 24, 2011 (ES) P201131705

(51) Int. Cl. A23P 1/04; A23L 1/305; A61K 47/42; A23J 3/14; A61K 9/50; A61K 9/00

(52) U.S. Cl. CPC A23P 1/04; A61K 9/5052; A61K 9/5036; A61K 9/5089; A61K 9/0092; A61K 47/42; A23J 3/14; A23L 1/3055; A23V 2002/00
USPC 424/491; 530/370; 514/773; 106/124.1; 106/135.1; 424/400; 264/10; 264/465; 264/555; 426/93

(57) ABSTRACT

The invention relates to micro-, submicro- or nano-structures comprising amaranth protein, optionally combined with at least one other biopolymer, which structures are suitable for use as an encapsulation matrix. In particular, the invention relates to micro-, submicro- or nano-structures comprising amaranth protein and a polysaccharide. The invention also relates to the production method thereof, said method comprising an electrospinning, electrospraying or blow spinning step. The encapsulated product is characterised in that it comprises an encapsulation matrix formed by micro-, submicro- or nano-structures of the invention and at least one functional ingredient. The invention further relates to the method for obtaining same.

### TECHNICAL FIELD

The present invention relates to the development of micro-, submicro- and nano-structures containing isolated amaranth protein and mixtures of isolated amaranth protein with other biopolymers, as well as its use in product encapsulation. The development of these encapsulation structures may be carried out by using any known technique for this purpose such as spray drying or atomisation, electrospinning or electrospraying, or blow-spinning or blow-spraying. These micro-, submicro- and nano-structures are intended for use in food, pharmaceutical and biomedical products and/or packages with a high potential in the field of encapsulation of added value compounds in these and other applications of interest. However, they may also be useful for encapsulating active ingredients in other fields such as agriculture, as well as the textile or shoe industry.

### STATE OF THE ART

Amaranth (Amaranthus hypocondriacus L., Revancha variety), is a pseudocereal that has grains and leaves with a high nutritional value. It is a traditional Mexican plant that offers wide advantages as it is easy to cultivate, although it is considered as an “underutilised crop”. Due to its agricultural and nutritional characteristics, amaranth has been considered by the U.S. National Academy of Science (NAS) and by the Food and Agriculture Organization of the United Nations (FAO), as a promising plant for economic development (National Academy of Science. 1975. Amaranth: Modern Prospects for an Ancient Crop; National Academy Press: Washington D.C., p. 189). Its protein content is generally higher than in other cereals such as wheat (Bressani, R., 1994. Composition and Nutritional Properties of Amaranth. In: Amaranth Biology, Chemistry and Technology, Paredes-Lopez, O. (Ed.). CRC Press, USA., pp: 185-205; Koziol, M. (1992). Chemical composition and nutritional evaluation of quinoa (Chenopodium quinoa Wild). Journal of Food Composition and Analysis, 5, 35-68). Amaranth grain has proteins such as globulins and albumins, and worthy of note is its low or zero content in prolamins, a feature that makes it a potential ingredient for people with celiac disease (Jerzy Drzewiecki, Efren Delgado-Licon, Ratiporn Haruenkit, Elke Pawelzik, Olga Martin-Belloso, Yong-Seo Park, Soon-Teck Jung, Simon Trakhtenberg and Shela Gorinstein. Identification and Differences of Total Proteins and Their Soluble Fractions in Some Pseudocereals Based on Electrophoretic Patterns. J. Agric. Food Chem. 2003, 51, 7798-7804; Gorinstein, S.; Pawelzik, E.; Delgado-Licon, E.; Haruenkit, R.; Weisz, M.; Trakhtenberg, S. Characterization of pseudocereal and cereal proteins by protein and amino acid analyses. J. Sci. Food Agric. 2002, 82, 886-891).

Due to the increase in the demand for new products with a high nutritional value, several studies have been undertaken concerning amaranth grain as a high-quality, promising crop, not only as an ingredient for food formulations, but also as a material for the development of edible and biodegradable films (Colla, E., P. J. Do Amaral Sobral and F. C. Menegalli, 2006. Amaranthus cruentus flour edible films: influence of stearic acid addition, plasticizer concentration and emulsion stirring speed on water vapor permeability and mechanical properties. J. Agricultural and Food Chemistry, 54(18):6645-6653; D. Tapia-Blácido, A. N. Mauri, F. C. Menegalli, P. J. A. Sobral, and M. C. Añon, 2007. Contribution of the Starch, Protein, and Lipid Fractions to the Physical, Thermal and Structural Properties of Amaranth (Amaranthus caudatus) Flour Films. Journal of Food Science, 72 (5), 293-300; D. Tapia-Blácido, P. J. Sobral and F. C. Menegalli. 2005. Development and characterization of biofilms based on Amaranth flour (Amaranthus caudatus). Journal of Food Engineering, 67, 215-223; N. J. Elizondo, P. J. Sobral and F. C. Menegalli. 2009. Development of films based on blends of Amaranthus cruentus flour and poly(vinyl alcohol). Carbohydrate Polymers, 75, 592-598).

Among the encapsulation techniques that are useful for the development of micro-, submicro- and nano-structures containing amaranth protein, electrospinning/electrospraying (high voltage spinning/spraying) and blow spinning/spraying stand out. These techniques are simple and highly versatile methods for obtaining fibres and/or capsules in the submicrometric range by means of the effects of an external electrical field applied between two electrodes in the case of electrospinning/electrospraying, and by means of fluid pressure in the case of blow spinning/spraying to which the polymeric solution is subjected. These processes do not require the use of temperature. In recent years, the use of these techniques has aroused considerable interest for the development of fibres from food grade biopolymers such as proteins and polysaccharides, opening up vast possibilities for the implementation of novel, renewable, edible and biodegradable materials in sectors as diverse as the food, pharmaceutical and biomedical sectors.

In the field of foodstuffs, the development of nanofibres from proteins such as zein, a protein isolated from soya and wheat protein, has already been reported (S. Torres-Giner, E. Gimenez, J. M. Lagaron. 2008. Characterization of the morphology and thermal properties of Zein Prolamine nanostructures obtained by electrospinning. Food Hydrocolloids, 22, 601-614; Woerdeman, D. L., P. Ye, S. Shenoy, R. S. Parnas, G. E. Wnek and O. Trofimova. 2005. Electrospun Fibers from Wheat Protein: Investigation in the Interplay between Molecular Structure and the Fluid Dynamics of the Electrospinning Process. Biomacromolecules. 6: 707-712; Vega-Lugo, A. C., L. T. Lim. 2009. Controlled release of ally! isothiocyanate using soy protein and poly(lactic acid) electrospun fibers. Food Research International. 42: 993-940). There are also studies about the encapsulation of antibacterial agents, such as allyl isothiocyanate, in isolated soy protein nanofibers, and antioxidants such as catechins and β-carotene in zein nanofibers (Vega-Lugo, A. C., L. T. Lim. 2009. Controlled release of allyl isothiocyanate using soy protein and poly(lactic acid) electrospun fibers. Food Research International. 42: 933-940; Fernandez, A., S. Torres-Giner and J. M. Lagaron. 2009. Novel route to stabilization of bioactive antioxidants by encapsulation in electrospun fibers of zein prolamine. Food Hydrocolloids. 23 (5): 1427-1432. 13-17).

There is currently no precedent in the scientific literature or patents concerning the processes for obtaining micro-, submicro- and nano-structures comprising amaranth protein as an encapsulating material. In this invention, the use of amaranth protein and mixtures thereof with other biopolymers for the encapsulation and protection of valuable ingredients is proposed, and the process for obtaining micro-, submicro- and nano-fibres and micro-, submicro- and nano-capsules containing amaranth protein is described.

### DESCRIPTION OF THE INVENTION

### Brief description of the invention

The present invention refers to the use of amaranth protein and mixtures thereof with other biopolymers for the development of micro-, sumbicro- and nano-structures, be they fibres or capsules. These generated micro-, submicro- and nano-structures may be used as an encapsulation matrix for functional or technologically valuable ingredients and additives to be incorporated, though not by way of limitation, into pharmaceutical or food preparations.

The incorporation of bioactive compounds such as vitamins, probiotics, bioactive peptides and antioxidants in systems based in amaranth protein provides a pathway for the development of, for example, novel functional foods that may provide physiological benefits or reduce the risk of disease. The use of these incorporation vehicles, apart from being useful as such for inclusion in different food/pharmaceutical matrixes or those of any other area that can benefit from this technology, offers the consumer an encapsulation matrix that also has an additional nutritional value as the composition of aminoacids present in amaranth proteins is of high quality (compared to other proteins such as maize zein), allowing for the development of encapsulation systems with bifunctional properties.

Furthermore, due to its low or zero content in prolamine, the micro-, submicro- and nano-structures comprising amaranth protein in the present invention are suitable for administration to people with celiac disease.

The micro-, submicro- and nano-structures comprising amaranth protein in the present invention may be obtained by using any known technique to this end such as spray-drying or atomisation, coacervation, interfacial polymerisation, ionic gelation, polymeric incompatibility, liposome entrapment, use of supercritical fluids, fluidised bed encapsulation, electrospinning, electrospraying or blow spinning or spraying.

One preferred embodiment of said encapsulation process is by means of spinning techniques (electrospinning, electrospraying, blow spinning and blow spraying) as they do not use temperature and allow for control over the morphology and size of the obtained structures.

In addition, it is necessary to highlight that as the components of these encapsulation systems are of natural origin, the stability of the properties of the biopolymeric solutions, in particular viscosity, varies over time. This parameter is crucial in electrospinning/electrospraying and blow spinning/blow spraying processes in order to maintain flow continuity throughout the process, as blockage formation at the end of the solution ejection syringe causes yield and production time losses, specifically in large scale processes. Compared to zein polymeric solutions, amaranth protein solutions and mixtures of amaranth protein with other biopolymers retain the ability to form fibres/capsules 72 hours after the solution has been prepared, as well as offerering a blockage-free flow throughout the process, which confers an advantage over the previously mentioned systems.

### Detailed Description of the Invention

The present invention, provides micro-, submicro- and nano-structures, characterised in that they comprise an amaranth protein isolate (hereinafter amaranth protein), combined or not with at least another biopolymer.

In the present invention micro-, submicro- and nano-structure means a structure with a size of 1 to 100 microns, smaller than a micron, and smaller than 100 nanometres in size, respectively. In the present invention, the preferred sizes of the micro-, submicro- and nano-structures are between 1-10 microns, 0.1-1 microns and 10-100 nanometres, respectively.

Furthermore, in the present invention the term amaranth protein means protein obtained from the pseudocereal amaranth.

In a preferred embodiment, the micro-, submicro- or nano-structures of the present invention are suitable for use as an encapsulation matrix.

In another preferred embodiment, the micro-, submicro- or nano-structures of the present invention may be shaped as a fibre or capsule.

In another preferred embodiment, the micro-, submicro- or nano-structures of the present invention comprise amaranth protein with a minimum protein content of 70%. Preferably, the minimum protein content is 85%.

The micro-, submicro- or nano-structures of the present invention may comprise at least one biopolymer combined with amaranth protein. This biopolymer may be a polysaccharide, a lipid, another protein, a biopolyester, or any combination thereof.

In another preferred embodiment, the micro-, submicro- or nano-structures as defined in the present patent application comprise amaranth protein and at least one polysaccharide. Preferably, the micro-, submicro- or nano-structures of the present invention comprise amaranth protein and a polysaccharide.

In an even more preferred embodiment, the polysaccharide is a glucan, it being especially preferred that the glucan be pullulan.

In a further preferred embodiment, the micro-, submicro- or nano-structures as described in the present patent application, preferably when they are suitable to use as an encapsulation matrix, are characterised in that the amaranth protein to biopolymer ratio is between 30:70 and 100:0. Preferably, this ratio is between 50:50 and 80:20.

In a further preferred embodiment, the micro-, submicro- and nano-structures suitable for use as an encapsulation matrix as described in the present patent application, are characterised in that the amaranth protein to polysaccharide ratio, preferably amaranth protein to pullulan, is between 30:70 and 100:0. Preferably, this ratio is between 50:50 and 80:20.

In another preferred embodiment, the micro-, submicro- or nano-structures of the present invention may further comprise one or more additives.

This additive may be used, for example, to enhance the properties of the micro-, submicro- or nano-structures, so as to facilitate the method for obtaining it, further processing thereof, or to modify the stability or release of the active ingredient that may be encapsulated within the micro-, submicro- and nano-structures of the invention.

Preferably, the additive comprised within, the micro-, submicro- or nano-structures as defined in the present patent application may be selected from among the group consisting of a surfactant (such as polyoxyethylene sorbitan monooleate (Tween 80), lecithin or sodium estearoyl lactate), a crosslinker (such as, for example, glutaraldehyde), fibres (such as cellulose fibres), non-amaranth protein (such as zein or whey proteins), a plasticiser (such as glycerol), an acid (such as citric acid), a base (such as sodium hydroxide), an emulsifier (such as polyoxyethylene monolaureate), an antioxidant (such as quercetin) and mixtures thereof.

In an even more preferred embodiment, when the additive comprised within the micro-, submicro- or nano-structures as defined in the present patent application is a fibre, said fibre is a cellulose fibre.

In an even more preferred embodiment, the additive is a surfactant, it being even more preferable that it be a polysorbate. In an especially preferable manner, the additive comprised within the micro-, submicro- or nano-structures of the present invention is polyoxyethylene sorbitan monooleate (commercially known as Tween 80).

The micro-, submicro- or nano-structures of the present invention may be obtained by means of any known technique to this end such as spray drying, coacervation, interfacial polymerisation, ionic gelation, polymeric incompatibility, liposome entrapment, and use of supercritical fluids. Likewise, the micro-, submicro- or nano-structures of the present invention may also be obtained by means of common encapsulation techniques such as fluidised bed, electrospinning or electrospraying and blow spinning or spraying.

Amaranth protein may be isolated from a commercial amaranth protein concentrate by using the isoelectric point precipitation method, for example, as described in the examples section of the present patent application.

In order to obtain micro-, submicro- and nano-structures from said protein isolate, a solution of amaranth protein in acidic solvent (for example: acetic, formic, hydrochloric acid, etc.) or in organic solvent (for example: hexafluoropropanol) may be prepared, to which other additives such as surfactants or other specific ingredients that enhance the protection of the encapsulation matrix (such as crosslinkers, polysaccharides, fibres, other proteins, etc.) may be added. The active ingredients to be encapsulated may also be incorporated to the solution, as long as they are soluble in said solvents, in order to obtain the encapsulated product.

Additionally, if the ingredients to be encapsulated are not soluble in the solvent used for amaranth protein, two solutions may be prepared: one with the protein isolate and another with the ingredient to be encapsulated. Afterwards, these solutions are pumped through different ducts to the micro-, submicro- or nano-structure forming site by any of the aforementioned methods.

In a second aspect, the present invention also provides a method for obtaining the micro-, submicro- or nano-structures of the present invention, preferably suitable to use as an encapsulation matrix, characterised in that it comprises an electrospinning, electrospraying or blow-spinning step.

In a preferred embodiment, the method for obtaining the micro-, submicro- or nano-structures of the present invention may comprise an additional previous step of preparing an amaranth protein solution in one or more suitable solvents. Preferably, this solution may further comprise at least another biopolymer and/or may comprise at least one additive as described in the present patent application.

Preferably, the amaranth protein solution in one or more suitable solvents may comprise a polysaccharide and/or a surfactant. Even more preferably, the polysaccharide is pullulan, the surfactant is polyoxyethylene sorbitan monooleate and the solvent is formic acid.

In another preferred embodiment, the method for obtaining the micro-, submicro- and nano-structures of the present invention may comprise the following steps:
a) preparing a solution comprising amaranth protein in one or more solvents,
b) feeding the atomisation, electrospinning, electrospraying or blow-spinning equipment with the solution prepared in step a).

In another preferred embodiment, the solution prepared in step a) additionally comprises at least another biopolymer and/or at least one additive as described in the present patent application.

The biopolymer may be a polysaccharide, a lipid, another protein, a biopolyester or any combination thereof. Preferably, the biopolymer is a polysaccharide. In an even more preferred embodiment, the polysaccharide is a glucan, it being especially preferable that the glucan be pullulan.

In addition, the additive may be, for example, for enhancing the properties of the micro-, submicro- or nano-structures, so as to facilitate the method for obtaining it, further processing thereof, or to modify the stability or release of the functional ingredient that may be encapsulated within the micro-, submicro- or nano-structures of the invention.

Preferably, the additive comprised in the solution in step a) of the method of the invention as described in the present patent application, may be selected from among the group consisting of a surfactant (such as polyoxyethylene sorbitan monooleate (Tween 80), lecithin or sodium estearoyl lactate), a crosslinker (such as, for example, glutaraldehyde), fibres (such as cellulose fibres), non-amaranth protein (such as zein or whey proteins), a plasticiser (such as glycerol), an acid (such as citric acid), a base (such as sodium hydroxide), an emulsifier (such as polyoxyethylene monolaureate), an antioxidant (such as quercetin) and a mixture thereof. In a more preferable manner, the additive is a surfactant, it being especially preferable that this additive be a polysorbate.

In another even more preferred embodiment, the solution prepared in step a) of the method for obtaining micro-, submicro- or nano-structures of this invention may additionally comprise at least one polysaccharide and/or at least one surfactant.

In a further preferred embodiment, step a) for preparing the solution comprising amaranth protein includes a homogenising treatment by stirring and/or ultrasound. In an even more preferred embodiment, the preparation of the solution comprising amaranth protein in one or more solvents, with or without at least one additional additive and/or another biopolymer, takes place by means of a homogenising treatment for 10-300 s at between 15 and 40° C.

The solvent used to prepare the amaranth protein solution as described in the present patent application may be an acidic solvent such as acetic, formic, ferulic or hydrochloric acid; or an organic solvent such as hexafluoropropanol. Preferably, this solvent is formic acid.

In a further preferred embodiment, the solution prepared in step a) of the method for obtaining micro-, submicro- or nano-structures as described in the present patent application comprises between 0.1 and 99% of amaranth protein. More preferably, between 3 and 20% by weight with respect to the volume of the solvent

In a further preferred embodiment, the solution prepared in step a) of the method for obtaining micro-, submicro- or nano-structures as described in the present patent application comprises between 0.1 and 99% of another biopolymer, expressed by weight of biopolymer and amaranth protein with respect to the volume of the solvent. More preferably, between 3 and 20% by weight of biopolymer and amaranth protein with respect to the volume of the solvent.

In a further preferred embodiment, the solution prepared in step a) of the method for obtaining micro-, submicro- or nano-structures as described in the present patent application, additionally comprises between 0.1 and 99% of a surfactant, expressed by weight of surfactant with respect to the weight of the amaranth protein in the solution. Preferably, the surfactant is polyoxyethylene sortbitan monooleate (commercially available as Tween 80).

In an even more preferred embodiment, the solution prepared in step a) of the method of the invention comprises between 5 and 30% by weight of a surfactant expressed with respect to the weight of amaranth protein in the solution. Preferably, the surfactant is polyoxyethylene sorbitan monooleate (commercially available as Tween 80).

In another preferred embodiment, the method for obtaining micro-, submicro- or nano-structures as described in the present patent application comprises a step b) of electrospinning, electrospraying or blow-spinning characterised in that the distance between capillary and support may be between 0.1 and 200 cm. Preferably, between 2 and 50 cm.

In another preferred embodiment, the method for obtaining micro-, submicro- or nano-structures as described in the present patent application comprises a step b) of electrospinning, electrospraying or blow-spinning characterised in that the deposition speed may be between 0.001 and 100 ml/h. Preferably, between 0.01 and 10 ml/h.

In another preferred embodiment, the method for obtaining micro-, submicro- or nano-structures as described in the present patent application, comprises a step b) of electrospinning, electrospraying or blow-spinning characterised in that the applied voltage may be between 0.1 and 1,000 kV. Preferably between 5 and 30 kV.

In another preferred embodiment, the method for obtaining micro-, submicro- or nano-structures as described in the present patent application, comprises a step b) of electrospinning, electrospraying or blow-spinning characterised in that the distance between capillary and support is between 2 and 50 cm, the deposition speed is between 0.01 and 10 ml/h and the applied voltage is between 5 and 30 kV.

In another preferred embodiment, the method for obtaining micro-, submicro- or nano-structures as described in the present patent application, may comprise the following steps:
a) preparing a solution comprising between 0.1 and 99%, by weight, with respect to the volume of the solvent, of amaranth protein in one or more solvents, and
b) feeding the electrospinning, electrospraying or blow-spinning equipment with the solution prepared in a), wherein the distance between capillary and support is between 0.1 and 200 cm, the deposition speed is between 0.001 and 100 ml/h and the applied voltage is between 0.1 and 1,000 kV.

In an even more preferred embodiment, the method for obtaining micro-, submicro- or nano-structures as described in the previous paragraph, is characterised in that the solution prepared in step a) may additionally comprise between 0.1 and 99% of a polysaccharide, expressed by weight of polysaccharide and amaranth protein with respect to the volume of the solvent, and/or between 0.1 and 99% of a surfactant, expressed by weight of surfactant with respect to the weight of amaranth protein in the solution. Preferably, the polysaccharide is pullulan and the surfactant is polyoxyethylene sorbitan monooleate (commercially available as Tween 80).

In another even more preferred embodiment, the method for obtaining micro-, submicro- or nano-structures as described in the present patent application, may comprise the following steps:
a) preparing a solution comprising between 3 and 20%, by weight, with respect to the volume of the solvent, of amaranth protein in one or more solvents, and
b) feeding the electrospinning, electrospraying or blow-spinning equipment with the solution prepared in step a), wherein the distance between capillary and support is between 2 and 50 cm, the deposition speed is between 0.01 and 10 ml/h and the applied voltage is between 5 and 30 kV.

In another even more preferred embodiment, the method for obtaining micro-, submicro- or nano-structures as described in the previous paragraph may comprise that the solution prepared in step a) additionally comprises between 3 and 20% of a polysaccharide, expressed by weight of polysaccharide and amaranth protein with respect to the volume of the solvent, and/or between 5 and 30% of a surfactant, expressed by weight of surfactant with respect to the weight of amaranth protein in the solution. Preferably, the polysaccharide is pullulan and the surfactant is polyoxyethylene sorbitan monooleate (commercially available as Tween 80).

In a third aspect, the present invention also refers to the use of micro-, submicro- or nano-structures as described in the present patent application as a microencapsulation matrix.

Thus, the encapsulation matrix formed by the micro-, submicro- or nano-structures as described in the present invention may be used to encapsulate a wide range of functional ingredients from the pharmaceutical, food, or any other sector that may benefit from this technology such as the textile or shoe industry (for example, to encapsulate scents), as well as the packaging industry.

In a preferred embodiment, the functional ingredient comprised within the encapsulation matrix formed by micro-, submicro- or nano-structures as described in the present patent application is a functional pharmaceutical or food ingredient.

In an even more preferred embodiment, the functional pharmaceutical ingredient may be an active compound along with the corresponding carriers, or an active ingredient such as an antioxidant, a scent, an antimicrobial, a bioactive peptide, a vitamin, a prebiotic, a probiotic, etc.

In another even more preferred embodiment, the functional food ingredient may be selected from among the group consisting of a vitamin, a probiotic, a prebiotic, an antimicrobial, a bioactive peptide, an antioxidant and a mixture thereof.

In a fourth aspect, the present invention also relates to an encapsulated product comprising a microencapsulation matrix formed by micro-, submicro- or nano-structures as described in the present patent application and at least one functional ingredient.

In a preferred embodiment, the encapsulated product is a part of a pharmaceutical composition or a food. Preferably, when the encapsulated product is a part of a pharmaceutical composition, said composition is suitable for oral administration. The use of amaranth protein in the encapsulation matrix provides an additional nutritional value to the encapsulated product due to the high quality composition of the aminoacids present in the amaranth proteins (compared to other proteins such as maize zein), providing an encapsulation matrix with bifunctional properties.

In a fifth aspect of the present invention, the encapsulated product comprising a microencapsulating matrix formed by micro-, submicro- or nano-structures as described in the present patent application and at least one functional ingredient, may be obtained by means of the same method described above in the present patent application for obtaining the micro-, submicro- or nano-structures, adding the functional ingredient to be encapsulated to the solution comprising amaranth protein.

If the ingredients to be encapsulated are not soluble in the employed solvent, two solutions may be prepared, one with the amaranth protein and the other with the ingredient to be encapsulated.

In a preferred embodiment, the method for obtaining the encapsulated product of the present invention may comprise the following steps:
a1) preparing a solution comprising amaranth protein in one or more solvents,
a2) preparing a solution comprising at least one functional ingredient in one or more solvents, which solvent or mixture of solvents is equal to or different than the one in solution a1), and
b) feeding the electrospinning, electrospraying or blow-spinning equipment simultaneously with the solutions prepared in a1) and a2).

Preferably, the solution prepared in steps a1) and/or a2), may further comprise at least another biopolymer and/or at least one additive as described in the present patent application. Preferably, the biopolymer is a polysaccharide and the additive is a surfactant. Even more preferably, the polysaccharide is pullulan and the surfactant is polyoxyethylene sorbitan monooleate (commercially available as Tween 80).

In another preferred embodiment, the method for obtaining the encapsulated product of the present invention may comprise the following steps:
a1) preparing a solution comprising between 0.1 and 99% by weight with respect to the volume of the solvent of amaranth protein in one or more solvents,
a2) preparing a solution comprising between 0.1 and 50% by weight with respect to the volume of the solvent of at least one functional ingredient in one or more solvents, wherein said solvent or mixture of solvents is equal to or different than the one in solution a1), and
b) feeding the electrospinning, electrospraying or blow-spinning equipment simultaneously with the solutions obtained in a1) and a2).

Preferably, the method for obtaining the encapsulated product as described in the previous paragraph, is characterised in that the solution prepared in steps a1) and/or a2) may additionally comprise between 0.1 and 99% of a polysaccharide, expressed by weight of polysaccharide and amaranth protein with respect to the volume of the solvent, and/or between 0.1 and 99% of a surfactant, expressed by weight of surfactant with respect to the weight of amaranth protein in the solution. Preferably, the polysaccharide is pullulan and the surfactant is polyoxyethylene sorbitan monooleate (commercially available as Tween 80).

In another preferred embodiment, the method for obtaining the encapsulated product of the present invention may comprise the following steps:
a1) preparing a solution comprising between 3 and 20%, by weight with respect to the volume of the solvent, of amaranth protein in one or more solvents,
a2) preparing a solution comprising between 0.01 and 50%, by weight with respect to the volume of the solvent, of at least one functional ingredient in one or more solvents, which solvent or mixture of solvents is equal to or different than the one of solution a1), and
b) feeding the electrospinning, electrospraying or blow-spinning equipment simultaneously with the solutions obtained in a1) and a2), wherein the distance between capillary and support is between 2 and 50 cm, the deposition speed is between 0.01 and 10 ml/h and the applied voltage is between 5 and 30 kV.

Preferably, the method for obtaining the encapsulated product as described in the previous paragraph, is characterised in that the solution prepared in steps a1) and/or a2) may additionally comprise between 3 and 20% of a polysaccharide, expressed by weight of polysaccharide and amaranth protein with respect to the volume of the solvent, and/or between 5 and 30% of a surfactant, expressed by weight of surfactant with respect to the weight of amaranth protein in the solution. Preferably, the polysaccharide is pullulan and the surfactant is polyoxyethylene sorbitan monooleate (commercially available as Tween 80).

In another preferred embodiment, the method for obtaining an encapsulated product as described in the previous paragraphs, is characterised in that, when the functional ingredient is soluble in the solvent or mixture of solvents of solution a1), a single solution a3) is prepared comprising the amaranth protein and the soluble active ingredient.

Preferably, the method for obtaining an encapsulated product as described in the previous paragraphs, is characterised in that this solution a3) may additionally comprise at least another biopolymer and/or at least one additive as described in the present patent application.

In another preferred embodiment, the step for preparing the solutions a1), a2) or a3) may be carried out via vigorous stirring so as to ease the dispersion of the ingredient to be encapsulated and/or of the additives in the protein matrix.

The following examples and drawings are provided by way of illustration, and are not intended to limit the present invention.

### BRIEF DESCRIPTION OF THE DRAWINGS

FIG. 1. Shows a Scanning Electron Microscopy (SEM) image of micro- and nano-capsules of amaranth protein obtained by means of the electrospraying technique from a solution of the amaranth protein isolate in formic acid using Tween 80 as surfactant.

FIG. 2. Shows the images obtained by Scanning Electron Microscopy (SEM) of the encapsulation structures obtained from amaranth protein:pullulan mixtures using the following ratios: (a) 50:50, (b) 60:40, (c) 70:30 and (d) 80:20.

FIG. 3. Shows an image obtained by optical microscopy using a fluorescence source of fibres of amaranth protein:pullulan (50:50) with encapsulated quercetin. It may be observed in the image that the quercetin is homogeneously distributed along the fibres.

FIG. 4. Shows the fluorescence presented by quercetin, incorporated within the fibres of amaranth protein:pullulan (80:20) after 50 hours of UV lighting.

### EXEMPLARY EMBODIMENTS OF THE INVENTION

The invention is below illustrated by means of several tests carried out by the inventors, and of note is the effectiveness of the method of the invention for obtaining nanostructures containing amaranth protein and mixtures thereof with other biopolymers, and which is capable of protecting several bioactive or functional ingredients.

### Amaranth Protein Isolation

Amaranth protein may be isolated from a commercial amaranth protein concentrate by using the isoelectric point precipitation method detailed as follows: the amaranth protein concentrate is suspended in water (10% weight/volume). Then, pH is adjusted to 9 with 2N of sodium hydroxide. The suspension is stirred for 30 minutes at room temperature and is spun down for 20 minutes at 9,000 g. The pH of the supernatant is adjusted to 5 with hydrochloric acid 2N and is centrifuged again at 9,000 g for 20 minutes at 4° C. The pellet obtained in this way is resuspended in water, neutralised with 0.1 N of sodium hydroxide and is subsequently lyophilised. The protein content determined after this process by using the Kjeldahl method is 85.47±0.023%.

### EXAMPLE 1

### Development of Micro-, Submicro- and Nano-Capsules Containing Amaranth Protein

This example describes a typical method for obtaining micro-, submicro- and nano-structures containing amaranth protein using the high-voltage electrospraying technique.

In a first step, the solution of amaranth protein isolate in formic acid is prepared. The concentration of the amaranth protein isolate is 10% by weight with respect to the volume of the solvent and is stirred at room temperature until a homogeneous solution is obtained. In this step the Tween 80 surfactant, used at a concentration of 20% by weight with respect to the weight of the protein used, is also added.

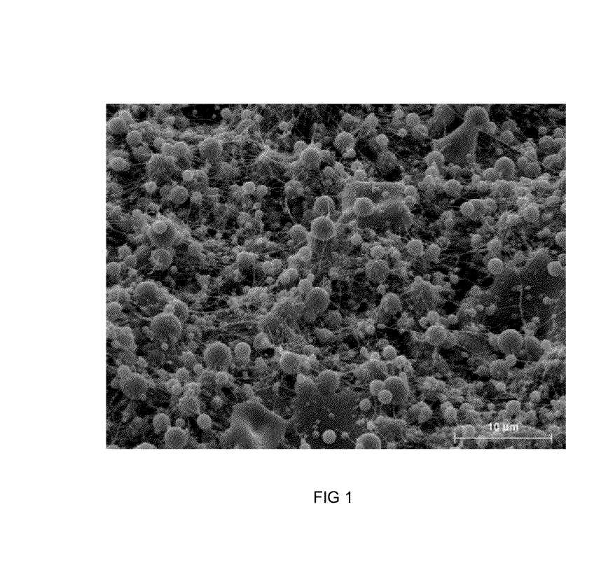

Once the solution is obtained, it is used to generate the nano-capsules by means of the electrospraying technique with a horizontal configuration. The solution is put into 5 ml syringes connected through Teflon tubes to a stainless steel needle with a diameter of 0.9 mm. The needle is connected to an electrode which is connected to a 0-30 kV power supply. A voltage of between 14-16 kV is applied and the solution is pumped through said needle at a flow of 0.4 ml/h. The counter-electrode is connected to a stainless steel plate (collector) wherein the obtained structures are collected, wherein the distance between the needle and the collector is approximately 10 cm. The process is carried out at room temperature. In this way, the nanocapsules shown in FIG. 1 are obtained.

The size of the microcapsules obtained in this way range between 90 nm and 3 microns, although microcapsules (i.e., capsules larger than 1 μm in size) are preferably obtained.

### EXAMPLE 2

### Development of Encapsulation Structures Using Amaranth Protein Mixtures and the Polysaccharide Pullulan

This example describes a typical method for obtaining micro-, submicro- and nano-fibres from mixtures of amaranth protein and a biopolymer (specifically, pullulan) by using the high-voltage electrospinning technique.

In a first step, the solution of amaranth protein isolate in formic acid is prepared. The concentration of the used amaranth protein isolate is about 10% by weight with respect to the volume of the solvent, and it is stirred at room temperature until a homogeneous solution is obtained. In this step, 10% by weight of the Tween 80 surfactant is also added and stirred until it is completely dissolved.

The biopolymer pullulan is then added at different concentrations, in such a way that the proportions of both polymers (amaranth protein:pullulan) varied as follows: 50:50, 60:40, 70:30 and 80:20, always maintaining the concentration of biopolymers in the solution at 20% (weight/weight). The mixtures were stirred at room temperature until a homogeneous mixture was obtained.

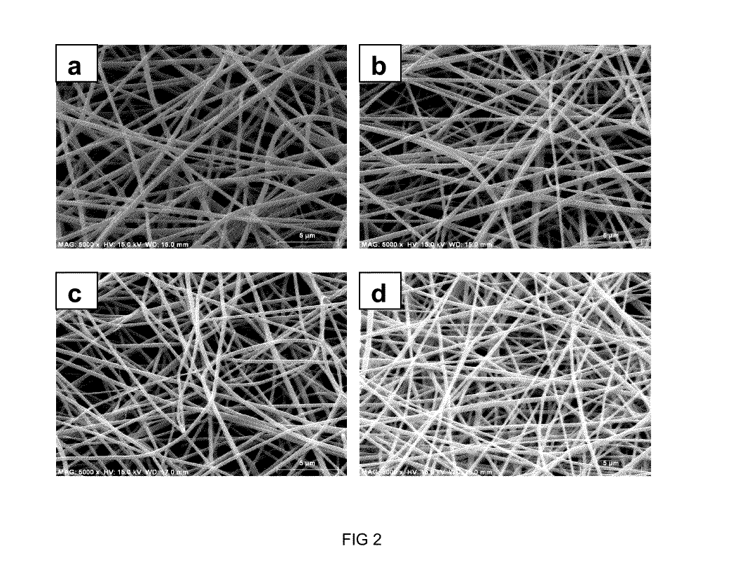

The second step of the process consisted of generating the encapsulation structures by using the high-voltage electrospinning technique under the following conditions: 15 kV voltage, 10 cm distance to the collector, 0.4 ml/h flow speed of the solution. The obtained morphologies are shown in FIG. 2. FIGS. 2a, 2b, 2c and 2d show the fibre morphologies obtained from amaranth protein:pullulan ratios of 50:50, 60:40, 70:30 and 80:20, respectively.

### EXAMPLE 3

### Quercetin Encapsulation in Fibres of Amaranth Protein/Pullulan

This example details the encapsulation process of the antioxidant quercetin in fibres developed from a mixture of amaranth protein/pullulan by using the high-voltage electrospinning technique.

In the first step of the process, a solution of amaranth protein and Tween 80 surfactant in formic acid is prepared. The concentration of the protein used is about 10% by weight with respect to the solvent, whereas the surfactant is used at a concentration of 10% with respect to the weight of the protein. This solution is stirred at 500 rpm at room temperature until a homogeneous mixture is obtained.

In a second step, pullulan is added at the same concentration as the amaranth protein in order to obtain a 50:50 mixture. Stirring continues at 500 rpm until the polysaccharide is completely incorporated into the dissolved mixture.

The third step consists of adding the quercetin antioxidant at a concentration of 10% with respect to the weight of amaranth protein and the mixture is stirred at room temperature until it is completely dispersed.

The obtained solution is put into a 5 ml plastic syringe for processing by means of the electrospinning process, under the following conditions: 15 kV voltage, 10 cm distance to the collector, 0.4 ml/h flow speed.

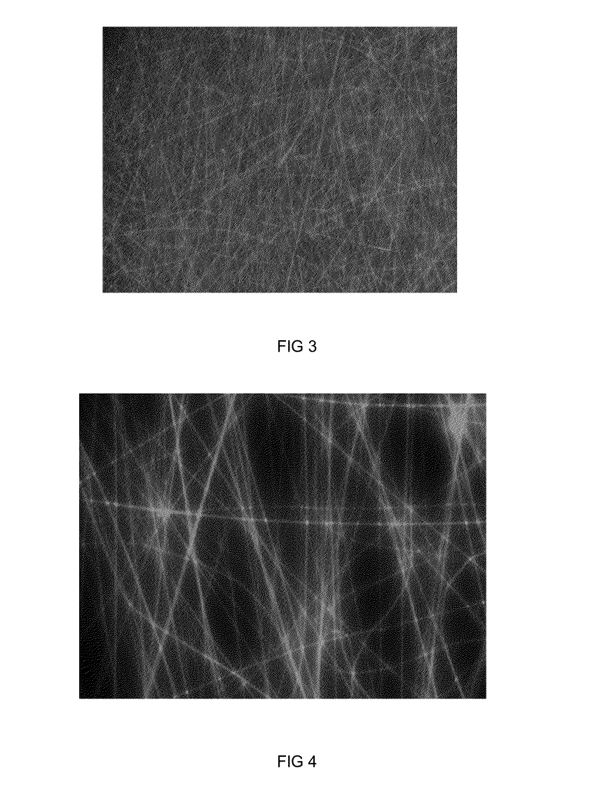

FIG. 3 shows the fluorescence displayed by quercetin, incorporated inside the fibres. A homogeneous distribution of the antioxidant inside the fibres is observed in the image.

### EXAMPLE 4

### Ferulic Acid Encapsulation in Fibres of Amaranth Protein/Pullulan

This example details the encapsulation process of ferulic acid, with antioxidant and anti-swelling properties, in fibres developed from a mixture of amaranth protein/pullulan by using the high-voltage electrospinning technique.

In the first step of the process a solution of amaranth protein:pullulan 80:20 in formic acid is prepared. The concentration of both biopolymers was 20% by weight with respect to the solvent. This solution was stirred at room temperature until a homogeneous mixture was obtained.

In a second step, the ferulic acid at a concentration of 20% with respect to the weight of the biopolymers is added, and the mixture is stirred at room temperature until it is completely dispersed.

The obtained solution is put into a 5 ml plastic syringe for processing by means of the electrospinning process, under the following conditions: 15 kV voltage, 10 cm distance to the collector, 0.4 ml/h flow speed.

The obtained fibres were subjected to prolonged UV light exposure so as to accelerate the photodegradation of the compound, and the encapsulation matrixes showed their ability to stabilise this bioactive compound. FIG. 4 shows the fluorescence shown by the ferulic acid, incorporated inside the fibres after 50 hours of UV exposure. In the images it is observed how after this prolonged exposure, the antioxidant still retains its fluorescence.

1. Micro-, submicro- or nano-structures comprising amaranth protein, combined or not with at least another biopolymer.

2. Micro-, submicro- or nano-structures according to claim 1, suitable for use as encapsulation matrixes.

3. Micro-, submicro- or nano-structures according to claim 1, wherein they are shaped as fibre or capsule.

4. Micro-, submicro- or nano-structures according to claim 1, wherein the biopolymer is a polysaccharide.

5. Micro-, submicro- or nano-structures according to claim 4, wherein the polysaccharide is pullulan.

6. Micro-, submicro- or nano-structures according to claim 1, wherein the amaranth protein to biopolymer ratio is between 30:70 and 100:0.

7. Micro-, submicro- or nano-structures according to claim 6, wherein the amaranth protein to biopolymer ratio is between 50:50 and 80:20.

8. Micro-, submicro- or nano-structures according to claim 1, wherein they comprise one or more additives.

9. Micro-, submicro- or nano-structures according to claim 8, wherein the additive is a surfactant.

10. Method for obtaining micro-, submicro- and/or nano-structures as described in claim 1, comprising an electrospinning, electrospraying or blow-spinning step.

11. Method for obtaining micro-, submicro- or nano-structures according to claim 10, wherein it comprises an additional previous step for preparing a solution of amaranth protein in one or more suitable solvents.

12. Method for obtaining micro-, submicro- or nano-structures according to claim 11, wherein the solution additionally comprises at least another biopolymer and/or at least one additive.

13. Method for obtaining micro-, submicro- or nano-structures according to claim 10, comprising the following steps:
a) preparing a solution comprising amaranth protein in one or more solvents,
b) feeding the electrospinning, electrospraying or blow-spinning equipment with the solution as prepared in step a).

14. Method for obtaining micro-, submicro- or nano-structures according to claim 13, wherein the solution prepared in step a) additionally comprises a polysaccharide and/or a surfactant.

15. Method for obtaining micro-, submicro- or nano-structures according to claim 13, comprising the following steps:
a) preparing a solution comprising between 0.1 and 99% by weight with respect to the volume of solvent, of amaranth protein in one or more solvents, and
b) feeding the electrospinning, electrospraying or blow-spinning equipment with the solution prepared in step a), wherein the distance between capillary and support is between 0.1 and 200 cm, the deposition speed is between 0.001 and 100 ml/h, and the applied voltage is between 0.1 and 1,000 kV.

16. Method for obtaining micro-, submicro- or nano-structures according to claim 15, wherein the solution prepared in step a) additionally comprises between 0.1 and 99% of a polysaccharide, expressed by weight of polysaccharide and amaranth protein with respect to the volume of the solvent, and/or between 0.1 and 99% of a surfactant, expressed by weight of surfactant with respect to the weight of amaranth protein in the solution.

17. Method for obtaining micro-, submicro- or nano-structures according to claim 15, comprising the following steps:
a) preparing a solution comprising between 3 and 20% by weight with respect to the volume of solvent, of amaranth protein in one or more solvents, and
b) feeding the electrospinning, electrospraying or blow-spinning equipment with the solution prepared in step a), wherein the distance between capillary and support is between 2 and 50 cm, the deposition speed is between 0.01 and 10 ml/h and the applied voltage is between 5 and 30 kV.

18. Method for obtaining micro-, submicro- or nano-structures according to claim 17, wherein the solution prepared in step a) additionally comprises between 3 and 20% of a polysaccharide, expressed by weight of polysaccharide and amaranth protein with respect to the volume of solvent, and/or between 5 and 30% of a surfactant, expressed by weight of surfactant with respect to the weight of amaranth protein in the solution.

19. Use of micro-, submicro- or nano-structures as described in claim 1, as an encapsulation matrix.

20. Encapsulated product comprising an encapsulation matrix formed by micro-, submicro- or nano-structures as described in claim 1, and at least one functional ingredient.

21. Encapsulated product according to claim 20, wherein the functional ingredient is a pharmaceutical or food ingredient.

22. Method for obtaining an encapsulated product as described in claim 20, comprising the following steps:
a1) preparing a solution comprising amaranth protein in one or more solvents,
a2) preparing a solution comprising at least one functional ingredient in one or more solvents, wherein said solvent or mixture of solvents is equal to or different to the one of solution a1), and
b) feeding the electrospinning, electrospraying or blow-spinning equipment simultaneously with the solutions prepared in a1) and a2).

23. Method for obtaining an encapsulated product according to claim 22, wherein when the functional ingredient is soluble in the solvent or mixture of solvents of solution a1), a single solution a3) is prepared, comprising the amaranth protein and the soluble active ingredient.

24. Method for obtaining an encapsulated product according to claim 22, wherein one or several of the solutions a1), a2) or a3) additionally comprise at least another biopolymer and/or at least one additive.
